(19) United States
(12) Patent Application Publication
(10) Pub. No.: US 2005/0177214 A1
(43) Pub. Date: Aug. 11, 2005

(54) Disposable cryotherapy device for the treatment of hemorrhoids with frozen healing media

(76) Inventor: Jerzy Pohler, Toronto (CA)

Correspondence Address: MARKS & CLERK, 1075 NORTH SERVICE ROAD WEST, SUITE 203, OAKVILLE, ON, L6M 2G2, CA

(21) Appl. No.: 11/052,110

(22) Filed: Feb. 8, 2005

Related U.S. Application Data
(63) Continuation-in-part of application No. 10/775,362, filed on Feb. 10, 2004.

(51) Int. Cl. A61F 7/00; A61F 7/12

(52) U.S. Cl. 607/105; 607/108; 607/113

(57) ABSTRACT

The present invention provides a method and devices for the treatment of hemorrhoids. The method involves the combined delivery of cryotherapy and medication therapy. The devices described provide cryotherapy for immediate relief of pain, itching, reduction of swelling, reduction of existing bleeding, shrinkage of hemorrhoidal tissue, and promotes healing of affective tissues. Subsequent medication release from these devices provides sustained healing and protracted relief of symptoms. The devices may be disposable and may be used externally or internally, and may include an invasive or non-invasive applicator.

### RELATED APPLICATIONS

This is a continuation in part application of U.S. patent application Ser. No. 10/775362, filed Feb. 10, 2004, the contents of which, are hereby incorporated by reference.

### FIELD OF THE INVENTION

The present invention relates to the field of devices and methods for the treatment of hemorrhoids, or the like.

### BACKGROUND OF THE INVENTION

Over ten million individuals in the United States suffer from painful hemorrhoids; a prevalence rate of more than 4%. It is therefore a significant medical problem.

The term hemorrhoid is usually used by the non-medical layperson to describe the symptoms caused by hemorrhoids, since hemorrhoids exist in all healthy persons. Hemorrhoids are clusters of vascular tissue (arterioles, venules, arterio-venular connections), smooth muscle and connective tissue. Most lay-people and physicians refer to hemorrhoids when these vascular cushions cause painful symptoms. Hemorrhoids may be external or internal. Internal types do not cause cutaneous pain but may prolapse and bleed and cause severe perianal pain through spasm induction of the sphincter complex or when strangulated and/or necrosed. Internal hemorrhoids may also produce pruritus (itching). External hemorrhoids induce pain through thrombosis (by straining, diarrhea, diet change). Pain results from the rapid distention of innervated sensitive tissue caused by both clot and edema (tissue swelling).

Currently accepted non-surgical medical treatments for the symptoms of hemorrhoids are directed at relieving the pain and/or pruritus (itching). Symptomatic relief should not only be directed towards the pain caused by thrombosis, prolapse and edema (swelling), however, but also to the pain resulting from spasm of the sphincter.

Therapies of the prior art include the use of cold to reduce edema and numb the anal region. Separately, the prior art also describes the use of analgesics and topical anaesthetics for pain relief, topical steroids or other anti-inflammatories, topical L-arginine for the relief of sphincter spasm (a major and common precipitant of pain), and stool softeners (longer term preventative therapy). No therapy in the prior art describes a treatment in which a cryotherapy and medication treatment are combined.

U.S. Pat. No. 3,939,842 issued to Harris on Feb. 24, 1976 teaches a frozen disposable insert as does this inventor's U.S. Pat. No. 4,844,073 issued to Pohler (Jul. 4, 1989). However, neither invention describes the use of medication in combination with cold or cryotherapy. The Harris device, as described in U.S. Pat. No. 4,563,182 (Jan. 17, 1986) to Stoy and Stoy, is of controversial utility given that it may produce “very low temperatures” and cause “frostbite”.

Further, U.S. Pat. No. 4,563,182 issued to Stoy and Stoy (“Stoy”) teaches a rectal insert comprising a probe produced entirely of a frozen gel (with or without medication). The device may be left in place for only 1-5 minutes after which the gel, and more importantly, the functional part of the rectal insert dissolves. This limits the therapy treatment to an acute 1-5 minute treatment after which the gel dissolves, the external portion falls away and the remaining dissolved liquid either moves out the anus or moves up the colon away from the affected area. Also, because the insert is made of a gel that may dissolve, the shape of the insert may change and thus become difficult to retain within the rectum during treatment. Alternatively, the shape of the insert may change in such a fashion that it is difficult to remove the insert from the rectum once the treatment is finished.

As such, in contrast to the present invention, Stoy merely provides a dissolvable device that is incapable of a protracted delivery of medication.

U.S. Pat. No. 969,134 issued to Cowie (“Cowie”) on Aug. 30, 1910, teaches the use of cooling to reduce hemorrhoidal swelling and pain. The patent teaches the implementation of a hollow tube filled with ice or other cooling medium. Cowie, however, does not speak to combining the therapy with medication delivery or speak to the issue of disposability.

U.S. Pat. No. 5,178,627 issued to Hudock (“Hudock”) teaches a rectal insert that delivers medicants through pores within the device. Hudock does not teach of a device that provides cryotherapy and does not teach a delay in the release of the medication as a result of the cryotherapy treatment.

U.S. Pat. No. 702,789, No. 1,271,456, No. 2,653,599, and No. 3,675,642, relate to devices used to expand or hold protruding veins and a priori teach a treatment differing from that described herein.

As such, while these prior art products have provided some advantages and improvement in the area of interest in the present application, it would still be desirable to provide further improvement therein.

### SUMMARY OF THE INVENTION

Accordingly, it is a principal advantage of the present invention to provide a device and a treatment method for providing a combined cyrotherapy and medication treatment wherein the medication treatment is preferably delayed until the cyrotherapy treatment has been initiated. As such, it would be desired to provide a unitary device that provides a cryotherapy effect prior to delivering a healing medium.

It is a further advantage of the present invention to provide a device that first provides a controlled cryotherapy effect and then provides a healing medium to hemorrhoids, rectal tissue after surgery, or the like, that would be useful and provide a combined therapeutic advantage that was not otherwise available.

It is a still further advantage of the present invention to provide a disposable device that provides a frozen medium and a healing medium, and that is both convenient and comfortable to use.

The advantages set out hereinabove, as well as other objects and goals inherent thereto, are at least partially or fully provided by the cyrotherapy device of the present invention, as set out herein below.

Accordingly, in one aspect, the present invention provides a combined cryotherapy and healing medium treatment device preferably for insertion into the rectum for the treatment of hemorrhoids or other such symptomatic conditions, comprising;
a body section which is preferably suitable for at least partial insertion into the rectum;
a wall section on at least a portion of said body section which wall section defines at least one cavity;
a healing medium located within said cavity; and
a sleeve having an open end such that said applicator can slide into said sleeve and said sleeve substantially covers said cavity so as to hold said healing medium within said cavity.

In the present invention, the cryotherapy treatment provides pain relief within minutes and initiates the reduction of the edema. After the cryotherapy treatment, a time-delayed medicant release occurs, delivering a healing medium, such as an analgesic, aneasthetic, antipruretic and/or spincter anti-spasmotic to the affect area, so as to provide longer term relief of pain, itching, and sphincter spasm, respectively.

In further aspect, the present invention also provides a method of using the device described herein.

As such, in its broadest aspects, a method and device are provided herein for dual cryotherapy and medicant delivery for the treatment of hemorrhoids or other anal conditions in which pain or other discomfort is involved. The treatment method may utilize a disposable or non-disposable device for its application, and treatment can be applied internal or externally of the rectum.

In its simplest embodiment the device includes a frozen active medical ingredient as a healing medium which is located within the cavity or cavities located on the wall of the device.

In the preferred embodiment the active medical ingredient is formulated to provide 5 or more minutes of cooling before melting or becoming a dispersible gel. As such, unlike solid devices, the healing medium, or medication, is initially trapped in cavities within the device and then slowly released from the cavities so as to be delivered over time as the healing medium warms.

The device is preferably substantially cylindrical in shape and has a substantially rounded insertion tip. The device is also preferably hollow so as to allow it to be filled with either a cryotheraputic material, and/or a healing medium, or a material which acts as both.

In a further preferred embodiment the device has a substantially bulbous portion along its length so as to stop disgorgement of said device. All embodiments preferably include an enlarged external portion (which can be round, oval, trapezo, or any other imaginable shape or design) which is suitable to essentially prohibit the device from entering entirely into the anal cavity, when the device is applied into the rectum to treat internal and/or external hemorrhoids.

In its more advanced embodiment the treatment method and device also allows for containment of a substance of high latent heat of fusion to be permanently sealed within the device. Such substances of high latent heat of fusion have the beneficial effect of providing cooling over an extended period of time. In this advanced embodiment, hemorrhoidal or other medication is contained primarily within external cavities of the device and released over time to the affected areas.

Whereas the prior art therapies provide either immediate, but brief, pain and/or pruritus relief (cryotherapy), or delayed pain and/or pruritus relief (medication), the method of the present invention provides the advantage that it provides both immediate and long-term symptom relief due to the combined cryotherapy treatment and subsequent treatment with the healing medium.

### BRIEF DESCRIPTION OF THE DRAWINGS

The above-mentioned, and other, features and advantages of this invention, and the manner of attaining them, will become apparent and be better understood by reference to the following description of the embodiments of the invention in conjunction with the accompanying drawings, wherein:

FIG. 1. is a plan view of the cryotherapy device of the present invention;

FIG. 2 is an exploded view of the device of FIG. 1;

FIG. 3 is a cross-sectional view of the device of FIG. 1;

FIG. 4 is a second plan view of the device of FIG. 1;

FIG. 5 is a partial cross-sectional view of the device of FIG. 1;

FIG. 6a is a perspective view of a second embodiment of the cryotherapy device of the present invention;

FIGS. 6b, 6c and 6d are a partial perspective view of a portion of the cryotherapy device of FIG. 6a, and a cross-sectional views thereof;

FIGS. 7a to 7d are plan and perspective views of a third embodiment of the cryotherapy device of the present invention; and

FIG. 8a and 8b display an example of the packaging for a device as described in FIG. 1.

### DETAILED DESCRIPTION OF THE PREFERRED EMBODIMENTS

The novel features which are believed to be characteristic of the present invention, as to its structure, organization, use and method of operation, together with further objectives and advantages thereof, will be better understood from the following drawings in which presently preferred embodiments of the invention will now be illustrated by way of example only. In the drawings, like reference numerals depict like elements.

It is expressly understood, however, that the drawings are for the purpose of illustration and description only and are not intended as a definition of the limits of the invention.

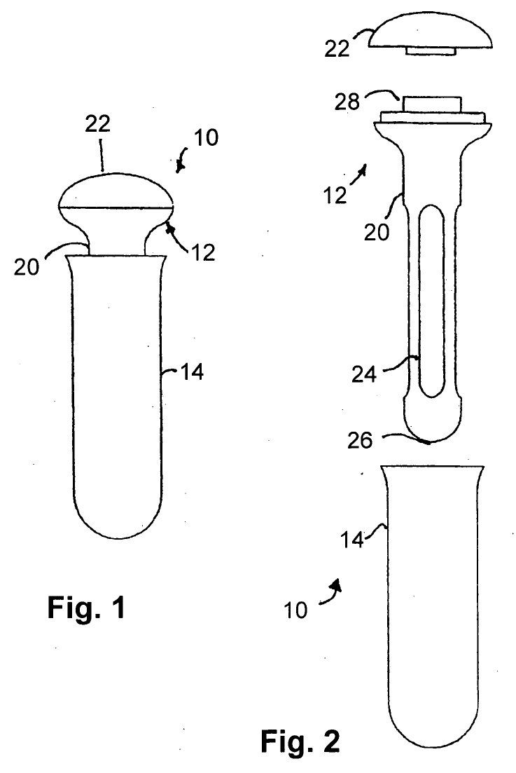

An example of a disposable cryotherapy device in accordance with the present invention, which is primarily intended for the treatment of hemorrhoids, rectal tissue after surgery, or the like, is shown in FIGS. 1 and 2. The cryotherapy device 10 includes a rectal insert 12 and a sleeve 14.

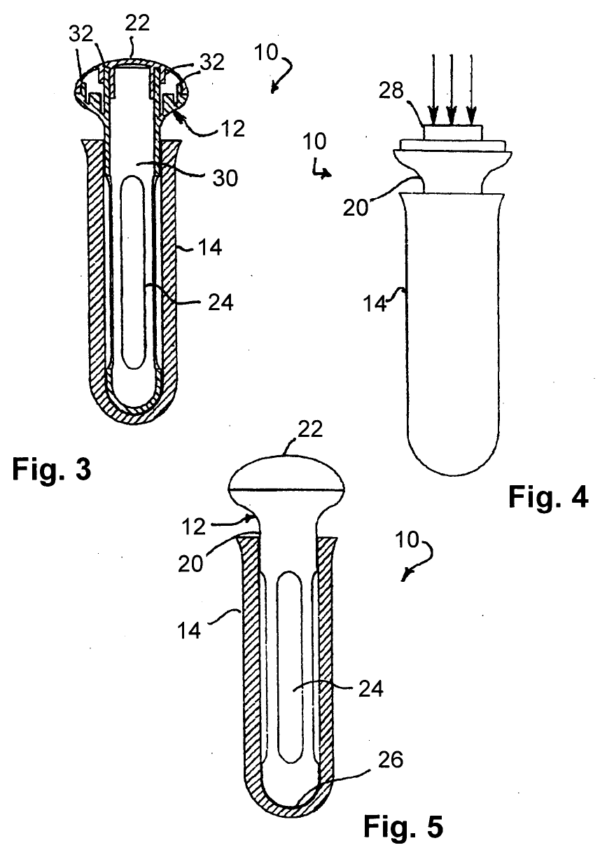

The rectal insert 12 includes a body 20 and a cap 22. In this example, the body 20 includes an external wall having cavities, or openings, in the forms of slots 24, a rounded tip 26, and a cap receiving portion 28. Body 20 encloses a chamber 30 which is shown in more detail in FIG. 3. The body 20 has a rounded tip 26, and a cylindrical shape for comfortable insertion into a rectum. The chamber 30 contains a frozen “active hemorrhoidal ingredient” (hereinafter “AHI”) which acts as a healing medium. The frozen AHI can be comprised of, for example, water, or a substance such as aloe vera gel mixed with purified water, glycerine, a carbomer and a sodium hydroxide solution. Alternatively, any existing hemorrhoid product such as, but not limited to licodaine, hydrocortisone, an appropriate analgesic, hamamelis water L-arginine or any other effective haemorrhoid medication when used either alone or mixed with water or a substance such as aloe vera gel mixed with purified water, or glycerine, or a carbomer and sodium hydroxide, might be used. It is to be understood, however, that AHI formulations with high heat capacity and high latent heats of fusion are preferred since they will provide superior cooling. It is also to be further understood that the construction -material and thickness of the device wall, and the selection of the AHI, are designed so that the conduction of heat away from the anus is limited and does not cause tissue freezing and necrosis.

To further maximize the cryotherapy treatment effect, the frozen AHI is preferably formulated such that it remains substantially “solid”, or frozen, for at least 1 minute, more preferably at least 2 minutes, and even more preferably at least 5 minutes after the cyrotherapy device is applied to the rectal tissue.

It is to be noted that the term “frozen” or “freezing” will be used hereinafter for brevity. However, it is to be clearly understood that these terms are to include any solidification, or any other suitable increase in viscosity, as a result of cooling, that permits the operation of the present invention.

It is preferred that the pH of the AHI be in a physiologically suitable range, and that the materials used are suitable for their intended application.

The cap 22 is configured for engaging the cap receiving portion 28 (FIG. 2) of the body 20. More particularly, the cap 22 is configured to snap onto the cap receiving portion 28 of the body 20 in a sealing manner as shown in FIG. 1 and FIG. 3. The cap 22 and the cap receiving portion 28 preferably include multiple complementary sealing structures 32 to retain the AHI within the chamber 30. Alternatively, cap 22 and cap receiving portion 28 may include complimentary threads to seal off the open end of the body 20.

The sleeve 14 fits and locks over the rectal insert 12 such that sleeve 14 completely cover the slots 24 as shown in FIGS. 3 and 5. In preparation for use, the user ensures that the body 20 of the rectal insert is fully inserted into the sleeve 14. The selected AHI is placed into the chamber 30 via the cap receiving portion 28, as indicated by the arrows shown in FIG. 4. The chamber 30 is preferably not completely filled to allow expansion of the AHI during changes in temperature. The cap 22 is then attached to the cap receiving portion 28. With this configuration, sleeve 14 allows the AHI to be placed into chamber 30 prior to being frozen by precluding the AHI from leaking out of slots 24 to any significant extent. Device 10 is then frozen.

After freezing, the device 10 may be used entirely externally wherein it is held between the buttocks, and as such, is non-invasive. Alternately, it may be inserted into the rectum and thus treat internal and external hemorrhoids. For some applications, such as pregnant and some post-operative patients, the device 10 can also be used with the sleeve 14 on by placing it between the buttocks, and thereby introduce cryotherapy treatment without medication.

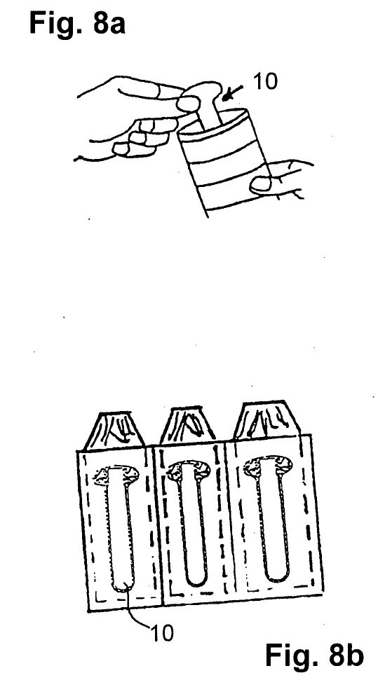

In order to treat hemorrhoids or rectal tissue after surgery with medication, however, the packaged, frozen cryotherapy device 10 is removed from the freezer and then separated from any packaging. The empty packaging, as shown in FIG. 8, is discarded. The rectal insert 12 is then removed from the sleeve 14 (FIG. 2). Again, as previously noted, the AHI does not leak through the slots 24 because it is now frozen. The rectal insert 12 is inserted into the rectum with the rounded end 26 first. The enlarged cap receiving 28 and cap 22 prevent the rectal insert 12 from being inserted so far into the rectum that it is difficult to remove.

Additionally, a string may be attached to the cap 22 to aid in the removal of the rectal insert 12 from the rectum.

During its use, the frozen AHI (and insert 12) first treats the tissue by cooling. Further, after several minutes, the frozen AHI begins to melt, and thus the AHI is delivered to the tissue providing a second phase of treatment which extends out over the time period of the AHI treatment. After treatment, the remaining rectal insert 12 is removed and it, sleeve 14, and the empty packaging are all discarded.

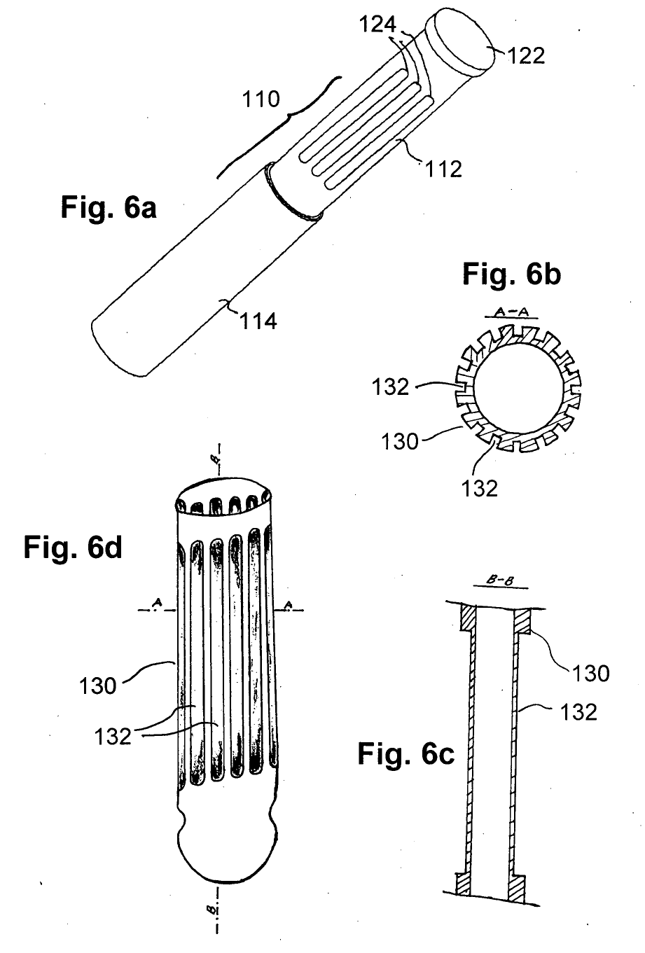

In a second embodiment, a non-invasive disposable or non-disposable cryotherapy device is shown. Also, in this embodiment, the device is configured so as to reduce the amount of medication required to fill the device which thereby reduces its cost and improving its utility. Such a device 110 is shown in FIG. 6a. In this embodiment the medication is placed in grooves 124 (FIG. 6a ) and there is no medication in the interior cavity of body 112. This reduces the amount of, and the cost of, medication required, as compared to device 10. The cryotherapy device 10 is configured to be applied externally to hemorrhoids and tissue and may be additionally used after surgery and includes a substantially cylindrical surface 112 having cavities in the form of slots 124 and a sleeve/cover 114. AHI is placed in the cavities 124 of the cryotherapy device and then frozen.

After freezing, the sleeve/cover 114 is removed and the substantially cylindrical surface 112 is positioned to contact either the external hemorrhoidal tissue. As such, a cryotherapy treatment is thus initially delivered, and as the AHI melts, prolonged relief is provided as the melted AHI exerts its effect on the tissue.

It should be noted that the configuration of the cryotherapy device 110 shown in FIG. 6a has four slots 124, however any number of slots or any other configuration of cavity or cavities may be used such as but not limited to concave dimples, holes, helical depressions, or grooves may be used to communicate the frozen AHI to the tissue and it is understood that many acceptable shapes may be imagined.

It is also to be understood that the device body 112 in FIG. 6a may be either solid or hollow.

In FIGS. 6b to 6d, a body section 130 is shown (in a perspective view (6c, and partial vertical (6d) and horizontal (6b) cross-sectional views) having a number of grooves 132 for receiving the AHI. It is further understood that if the body is hollow, as shown in FIG. 6c, the body 130 may contain any additional substance with the correct properties so as to improve the cryotherapy effect of the device.

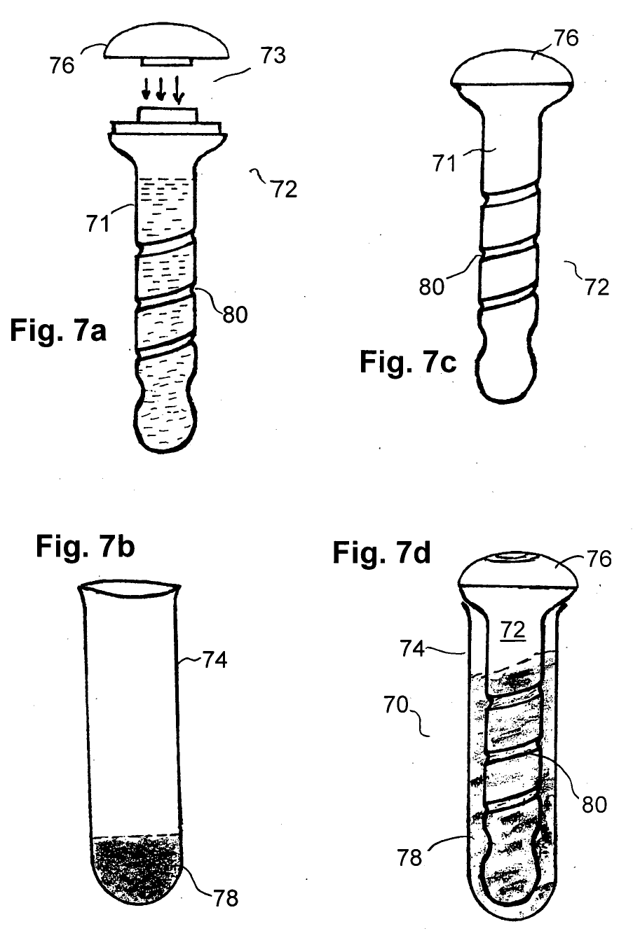

In a third embodiment of the invention, device 70 of FIG. 7 is designed so that an alternative method for placing the AHI into the device is proved. In FIG. 7a, the rectal insert portion 71 of body 72 of the device 70, which rectal insert portion 70 is hollow, is filled with a cryotherapy media through its opening 73, as indicated by the arrows, and as previously described, and then sealed with cap 76.

Unfrozen AHI 78 is dispensed into a sleeve 74 (FIG. 7b). The assembled insert 72, shown in FIG. 7c, is then inserted into sleeve 74 so as to push the AHI up the sides of body 72 and into the spiral cavity, or groove, 80 as shown in FIG. 7d. The completed device 70 is then frozen prior to use.

Alternatively, body 72 may be frozen prior to insertion into sleeve 74 in order to assist in freezing the AHI material.

The production method of FIG. 7 has the improved utility of allowing rapid manufacture of this device in terms of the step in which AHI is applied to the device, and in terms of the amount of AHI used. Further utility is gained by its use of generally transverse running grooves, such as, for example, spiral grooves, or by the use of other transverse running cavities, that assist in preventing the inadvertent release of the AHI when the patient removes the body section from the protective sleeve. Also, it is noted that the space between sleeve 74 and body 72 permits an increased amount of AHI to be attached to the exterior of body 72.

In its intended use the device is assembled, packaged and given to the patient as in the configuration of FIG. 7d. The patient then freezes this and once frozen, removes it from the packaging, removes the exterior sleeve (FIG. 7b) and inserts the body 72 so as to contact the hemorrhoidal tissue.

In a preferred embodiment, cap 76 and body 72 (FIG. 7) are manufactured so as to allow snap-together assembly as shown in FIG. 7a. Body 72 also preferably is provided so as to allow snap-together assembly with sleeve 74. This has the utility in that the additional frozen, or unfrozen AHI 78 (shown between the sleeve and probe) is sealed within the device.

The AHI used in this third embodiment could be as simple as water, or a substance such as aloe vera gel mixed with purified water, glycerine, a carbomer and a sodium hydroxide solution. Alternatively, an existing hemorrhoid product such as but not limited to licodaine, hydrocortisone, an appropriate analgesic, hamamelis water L-arginine or any other effective hemorrhoid medication is used either alone or mixed with water, or a substance such as aloe vera gel mixed with purified water, or glycerine, or a carbomer and a sodium hydroxide. Also, the AHI might be a commercial product such as Preparation H (tm), anusol, paceonia, or any other know suitable substance or natural healing media.

The packaged cryotherapy devices containing the AHI, as described in the present invention, are preferably placed in a freezer for at least at one hour to thereby freeze the AHI. The time required for freezing may, however, vary according to the formulation and the amount of the frozen AHI.

In use, the frozen AHI aids in assisting the treatment of the tissue by cooling the tissue. Further, after several minutes, the frozen AHI melts, and as such, the AHI is delivered to the tissue providing the second phase of the treatment, which extends out over the desired AHI treatment time period. After treatment, the rectal insert is removed from the rectum, and the insert, the sleeve, and the empty package are all discarded.

The cryotherapy device is preferably individually packaged as shown in FIGS. 8a and 8b. The packages may be sealable plastic bags or a series of blow moulded compartments sealed by conventional packaging methods that allows the compartments to open individually. Alternatively, the cryotherapy device may be packaged in a bubble wrap or any other packing method that keeps the device substantially sterile while in storage or during the freezing process.

While the invention has been described with reference to preferred embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

Thus, it is apparent that there has been provided, in accordance with the present invention, a combined cryotherapy and healing medium treatment device which fully satisfies the goals, objects, and advantages set forth hereinbefore. Therefore, having described specific embodiments of the present invention, it will be understood that alternatives, modifications and variations thereof may be suggested to those skilled in the art, and that it is intended that the present specification embrace all such alternatives, modifications and variations as fall within the scope of the appended claims.

Additionally, for clarity and unless otherwise stated, the word “comprise” and variations of the word such as “comprising” and “comprises”, when used in the description and claims of the present specification, is not intended to exclude other additives, components, integers or steps.

Moreover, the words “substantially” or “essentially”, when used with an adjective or adverb is intended to enhance the scope of the particular characteristic; e.g., substantially planar is intended to mean planar, nearly planar and/or exhibiting characteristics associated with a planar element.

Further, use of the terms “he”, “him”, or “his”, is not intended to be specifically directed to persons of the masculine gender, and could easily be read as “she”, “her”, or “hers”, respectively.

Also, while this discussion has addressed prior art known to the inventor, it is not an admission that all art discussed is citable against the present application.

1. A combined cryotherapy and healing medium treatment device for the treatment of hemorrhoids or other such symptomatic conditions, comprising;
a body section;
a wall section on at least a portion of said body section which wall section defines at least one cavity;
a healing medium located within said cavity; and
a sleeve having an open end such that at least a portion of said body section can slide into said sleeve so that said sleeve substantially covers said cavity and holds said healing medium within said cavity.

2. A device as claimed in claim 1 wherein said body section is hollow and thereby defines a hollow section, and said cavity is a slot which extends through said wall into said hollow section so that said healing medium is located within said slot and said hollow section.

3. A device as claimed in claim 1 wherein said cavity is a groove located on said wall section.

4. A device as claimed in claim 3 wherein said groove is a transverse running groove.

5. A device as claimed in claim 4 wherein said groove is a helical groove extending around said applicator.

6. A device as claimed in claim 1 having a plurality of cavities, and said cavities provide a plurality of slots, concave dimples or grooves on said wall portion.

7. A device as claimed in claim 1 wherein said healing medium is, in use, a frozen liquid or a chilled gel located within said cavity.

8. A device as claimed in claim 2 wherein said healing medium is, in use, a frozen gel or chilled liquid located within a slot in said wall portion, and also located within said hollow section of said applicator.

9. A device as claimed in claim 1 wherein said healing medium is chilled so as to provide a cyrotherapy treatment.

10. The device as claimed in claim 2 wherein said body section comprises a sealing cap and a rectal insert.

11. A device as claimed in claim 10 wherein said sealing cap and said rectal insert have a plurality of complementary sealing structures.

12. A device as claimed in claim 1 wherein said sleeve is sealed to said body section by a complementary sealing structure.

13. A device as claimed in claim 1 wherein said body section is substantially cylindrical in shape and has a rounded first end for insertion into the rectum.

14. A device as claimed in claim 13 wherein said body section comprises an enlarged second end to prevent said body section from being fully inserted into the rectum.

15. A device as claimed in claim 14 wherein said enlarged second end of said body section comprises a sealing cap.

16. A device as claimed in claim 1 wherein said device is individually packaged, and is disposable after use.

17. A device as claimed in claim 1 wherein said healing medium is an analgesic, anaesthetic antipruretic or sphincter anti-spasmodic medication.

18. A device as claimed in claim 1 wherein said healing medium is an active hemorrhoidal ingredient (AHI)

19. A device as claimed in claim 18 wherein said AHI is a substance known to be of benefit in the treatment of hemorrhoids.

20. A device as claimed in claim 18 wherein said AHI is water, aloe vera gel mixed with water, glycerine, a carbomer and a sodium hydroxide solution.

21. A device as claimed in claim 18 wherein said AHI is licodaine, hydrocortisone, an appropriate analgesic, or hamamelis water L-arginine, used either alone or mixed with water, aloe vera gel mixed with water, glycerine, or a carbomer and sodium hydroxide solution.

22. A device as claimed in claim 18 wherein the delivery of the AHI from said healing medium is substantially delayed for a period of at least 1 minute after insertion into the rectum.

23. A device as claimed in claim 18 wherein the delivery of the AHI from said healing medium is substantially delayed for a period of at least 5 minutes after insertion into the rectum.

24. A device as claimed in claim 1 wherein the healing medium is formulated to also provide the cryotherapy effect.

25. A device as claimed in claim 24 wherein said healing medium has a high heat capacity and high latent heat of fusion.

26. A method for the treatment of hemorrhoids, or other such symptomatic conditions of the rectum, comprising treating the affected area of the rectum with both cryotherapy and medication therapy, which therapies are provided by a combined cryotherapy and healing medium treatment device for the treatment of hemorrhoids or other such symptomatic conditions, comprising;
a body section;
a wall section on at least a portion of said body section which wall section defines at least one cavity;
a healing medium located within said cavity; and
a sleeve having an open end such that at least a portion of said body section can slide into said sleeve so that said sleeve substantially covers said cavity and holds said healing medium within said cavity are delivered by said treatments, wherein said therapy comprises:
cooling said combined cryotherapy and healing medium treatment device to a temperature wherein said healing medium is a solid or a gel;
removing said body section from said sleeve;
inserting a portion of said body section into the rectum, or the affected rectal area;
allowing said body section to be heated by the body temperature of the user so as to increase the temperature of said healing medium to a point wherein it liquifies and provides a medication therapy; and
removing said combined cryotherapy and healing medium treatment device from said rectum or rectal area.

27. A method as claimed in claim 26 wherein said method is used to treat pre- and postpartum pain and post-surgical pain of the rectum.

28. A method as claimed in claim 26 wherein said cryotherapy provides very prompt symptom relief, and the medication contained in said healing medium provides extended symptom relief once the effect of the cryotherapy has worn off.

29. A method for production of a combined cyrotherapy and medication therapy, which therapies are provided by a combined cryotherapy and healing medium treatment device for the treatment of hemorrhoids or other such symptomatic conditions, comprising;
a body section;
a wall section on at least a portion of said body section which wall section defines at least one cavity;
a healing medium located within said cavity; and
a sleeve having an open end such that at least a portion of said body section can slide into said sleeve so that said sleeve substantially covers said cavity and holds said healing medium within said cavity are delivered by said treatments, wherein said production method comprises:
providing a body section having a wall section defining said cavity;
inserting said body section into said sleeve; and
dispensing healing medium into said cavity.

30. A method as claimed in claim 29 wherein said healing medium is inserted into said sleeve before said body section is inserted into said sleeve.

31. A method as claimed in claim 29 wherein said body section is hollow, said healing medium is also dispensed within said hollow body section, and said hollow section is subsequently closed by affixing a cap to said body section.

32. A method as claimed in claim 31 wherein said cap and said body section have a plurality of complementary sealing structures.
